(19) United States
(12) Patent Application Publication
(10) Pub. No.: US 2019/0343959 A1
(43) Pub. Date: Nov. 14, 2019

(54) Biocampatible and Biodegradable Anionic Hydrogel System

(71) Applicant: BOARD OF TRUSTEES OF THE UNIVERSITY OF ARKANSAS, Fayetteville, AR (US)

(72) Inventors: Jingyi Chen, Fayetteville, AR (US); Tengjiao Wang, Fayetteville, AR (US); Isabelle Niyonshuti, Fayetteville, AR (US); Suresh Kumar Thallapuranam, Fayetteville, AR (US); Ravi K. Gundampati, Fayetteville, AR (US); Shilpi Agrawal, Fayetteville, AR (US); Kyle P. Quinn, Fayetteville, AR (US); Jake D. Jones, Fayetteville, AR (US)

(73) Assignee: BOARD OF TRUSTEES OF THE UNIVERSITY OF ARKANSAS, Fayetteville, AR (US)

(21) Appl. No.: 16/409,495

(22) Filed: May 10, 2019

Related U.S. Application Data
(60) Provisional application No. 62/670,578, filed on May 11, 2018.

(51) Int. Cl. A61K 47/58; A61K 47/69; A61K 9/00; A61K 9/06; A61K 38/18; A61P 17/02; C08F 283/06

(52) U.S. Cl. CPC A61K 47/58; A61K 47/6903; A61K 9/0019; C08F 283/065; A61K 9/0024; A61K 38/1825; A61P 17/02; A61K 9/06

(57) ABSTRACT

An anionic hydrogel based on poly(acrylic acid)-co-poly(oligoethylene glycol monoacrylate) (PAA-co-POEGA) for protein delivery and method of making the same.

### RELATED APPLICATIONS

This application claims priority to U.S. Provisional Application Ser. No. 62/670,578 filed on May 11, 2018, which is hereby incorporated in its entirety

### STATEMENT REGARDING FEDERALLY SPONSORED RESEARCH & DEVELOPMENT

Not applicable.

### INCORPORATION BY REFERENCE OF MATERIAL SUBMITTED ON A COMPACT DISC

Not applicable.

### BACKGROUND OF THE INVENTION

Hydrogels are three-dimensional, cross-linked polymer networks that can retain a large amount of water. They allow biomolecules to be trapped in their porous structures for delivery in applications such as tissue engineering and wound healing. In general, hydrogels can be made from both natural and synthetic polymers. Although natural polymers are often biocompatible and biodegradable, hydrogels made of natural polymers are difficult to be chemically functionalized for sustained release and/or potentially immunogenic in the host body. In contrast, hydrogels made from synthetic polymers have several advantages including modifiable chemical properties, tunable mechanical properties, controllable porosity and transport properties. Poly(ethylene glycol) (PEG) is commonly used to form hydrogels due to its good biocompatibility and non-immunogenity; however, PEG hydrogels typically possess minimal or no intrinsic biological activity due to the antifouling nature of the PEG polymers.

### BRIEF SUMMARY OF THE INVENTION

In other embodiments, the present invention provides a method, system, approach and solution that provide biocompatible and biodegradable hydrogels for sustained delivery of biological therapeutic agents.

In other embodiments, the present invention provides a system, method, approach and solution that provide biocompatible and biodegradable hydrogels for regenerative medicine applications.

In other embodiments, the present invention provides a system, method, approach and solution that provide a method to synthesize biocompatible and biodegradable anionic hydrogels based on poly(acrylic acid)-co-poly(oligoethylene glycol monoacrylate) for protein delivery.

In other embodiments, the present invention provides a system, method, approach and solution that provide a synthesis that involves an aqueous free radical polymerization.

In other embodiments, the present invention provides a system, method, approach and solution that provide a synthesis that involves an aqueous free radical polymerization, followed by sequential steps to allow the swelling of copolymer into the hydrogels.

In other embodiments, the present invention provides a system, method, approach, and solution wherein the introduction of an anionic group-containing acrylic acid into the copolymers changes the thermal properties and viscosity of the hydrogels due to the alternation of intermolecular interactions in the polymer networks. In these embodiments, electrostatic interaction(s) between the anionic hydrogels and positively charged proteins render the hydrogels capable of a sustainable release of the proteins.

In other embodiments, the present invention provides a system, method, approach and solution that provide an ionizable component, acrylic acid (AA), into the PEG-based hydrogel to form a biocompatible and biodegradable, anionic hydrogel of poly(acrylic acid)-co-poly(oligoethylene glycol monoacrylate) (PAA-co-POEGA).

In other embodiments, the present invention provides a system, method, approach and solution that provide a synthesis that involve an aqueous free radical polymerization, followed by sequential steps to allow the swelling of copolymer into the hydrogels. The presence of AA provides a negative charge in the hydrogel at physiological pH that improves the retention of the entrapped, positively-charged proteins through electrostatic interactions for sustained delivery. PAA mimics heparin which possesses antithrombin-activating properties and may promote anti-inflammatory processes.

In other embodiments, the present invention provides a system, method, approach and solution wherein monomers AA and PEG acrylate (OEGA) may be polymerized by a cross-linker such as N,N′-methylenebis(acrylamide) (MBAm) or N,N′-bis(acryloypcystamine (BAC). Depending on the ratio of AA/OEGA/cross-linker, the chemical and physical properties of the resulting hydrogel may be tuned to facilitate the protein delivery for different applications.

In other embodiments, the present invention provides a system, method, approach and solution wherein the sustained release of proteins from the hydrogel may be attained by using both the model protein lysozyme and wild-type fibroblast growth factor 1 (wtFGF1) that are positively-charged under the physiological condition.

In other embodiments, the present invention provides a system, method, approach and solution wherein the injectable PAA-co-POEGA hydrogel is biocompatible. The biodegradability of the hydrogel may be attained by hydrogel cross-linking BAC.

In other embodiments, the present invention provides a method, system, approach and solution that provide biocompatible and biodegradable hydrogels for sustained delivery of biological therapeutic agents.

In other embodiments, the present invention provides a method, system, approach and solution that provide biocompatible and biodegradable hydrogels for regenerative medicine applications.

In other embodiments, the present invention provides a method, system, approach and solution that provide a method to synthesize biocompatible and biodegradable anionic hydrogels based on poly(acrylic acid)-co-poly(oligoethylene glycol monoacrylate) for protein delivery.

In other embodiments, the present invention provides a method, system, approach and solution that involves an aqueous free radical polymerization, followed by steps to allow the swelling of copolymer into the hydrogels.

In other embodiments, the present invention provides a method, system, approach and solution wherein the introduction of an anionic group-containing acrylic acid into the copolymers changes the thermal properties and viscosity of the hydrogels due to the alternation of intermolecular interactions in the polymer networks.

In other embodiments, the present invention provides a method, system, approach and wherein there is an electrostatic interaction(s) between the anionic hydrogels and positively charged proteins, thus, rendering the hydrogels capable of a sustainable release of the proteins.

In other embodiments, the present invention provides a method, system, approach and solution that provide an ionizable component, acrylic acid (AA), into the PEG-based hydrogel to form a biocompatible and biodegradable, anionic hydrogel of poly(acrylic acid)-co-poly(oligoethylene glycol monoacrylate) (PAA-co-POEGA).

In other embodiments, the present invention provides a method, system, approach and solution that involve an aqueous free radical polymerization, followed by sequential steps to allow the swelling of copolymer into the hydrogels.

In other embodiments, the present invention provides a method, system, approach and solution wherein the presence of AA provides a negative charge in the hydrogel at physiological pH that improves the retention of the entrapped, positively-charged proteins through electrostatic interactions for sustained delivery.

In other embodiments, the present invention provides a method, system, approach and solution wherein PAA mimics heparin which possesses antithrombin-activating properties and may promote anti-inflammatory processes.

In other embodiments, the present invention provides a method, system, approach and solution wherein the hydrogel synthesis consists of a two-step procedure involving a free radical polymerization to form a copolymer network followed by allowing the copolymer network swelling into hydrogel.

In other embodiments, the present invention provides a method, system, approach and solution wherein monomers AA and PEG acrylate (OEGA) may be polymerized by a cross-linker such as N,N′-methylenebis(acrylamide) (MBAm) or N,N′-bis(acryloyl)cystamine (BAC).

In other embodiments, the present invention provides a method, system, approach and solution wherein, depending on the ratio of AA/OEGA/cross-linker, the chemical and physical properties of the resulting hydrogel may be tuned to facilitate the protein delivery for different applications.

In other embodiments, the present invention provides a method, system, approach and solution wherein the sustained release of proteins from the hydrogel may be attained by using both the model protein lysozyme and wild-type fibroblast growth factor 1 (wtFGF1) that are positively-charged under the physiological condition.

In other embodiments, the present invention provides a method, system, approach and solution wherein the injectable PAA-co-POEGA hydrogel is biocompatible. The biodegradability of the hydrogel may be attained by hydrogel cross-linking BAC.

In other embodiments, the present invention provides a method, system, approach and solution wherein the synthesis of the injectable hydrogels consists of a two-step procedure, the initial step involves an aqueous free radical polymerization of AA and OEGA (M.W. 480) with a cross-linking agent such as MBAm or BAC and the second step is to let the PAA-co-POEGA polymeric network swell in buffered solution such as a saline solution with 0.1 wt. % Borax (antibacterial agent), forming the injectable hydrogels.

In other embodiments, the present invention provides a method, system, approach and solution wherein the reaction may be initiated by APS to form PAA-co-POEGA polymeric network.

It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

In the drawings, which are not necessarily drawn to scale, like numerals may describe substantially similar components throughout the several views. Like numerals having different letter suffixes may represent different instances of substantially similar components. The drawings illustrate generally, by way of example, but not by way of limitation, a detailed description of certain embodiments discussed in the present document.

FIG. 1 shows the synthesis of the injectable hydrogels based on the copolymer networks of PAA-co-POEGA for an embodiment of the present invention.

FIG. 2 is a plot of the release curve of wtFGF1 and shaped hydrogel of the embodiment of the present invention.

FIG. 3 shows the lysozyme released profile as a function of time for several embodiments of the present invention.

FIG. 4 shows the loading capacity of lysozyme in the PAA-co-POEGA for various embodiments of the present invention at various AA concentrations (0, 0.5, 1, 1.5, 2 M) in DPBS (pH 7.4) at room temperature for 3 days.

FIG. 5A illustrates a loading and release study of the disk hydrogel of PAA-co-POEGA (1:1) with loading capacity of wtFGF1 as a function of time.

FIG. 5B illustrates a loading and release study of the disk hydrogel of PAA-co-POEGA (1:1) with wtFGF1 released from the hydrogel disk under different temperatures in DPBS(pH 7.4).

FIG. 6 is an in vivo biocompatibility study using a mouse model for GEL900 (left), 3M Telladerm Gel (middle), and no treatment(right) with the wound size being monitored for 10 days.

FIG. 7 illustrates the released wtFGF1 as a function of time for GEL900 and GEL800.

FIG. 8A illustrates a cell proliferation assay of the released wtFGF1 from GEL900 and GEL800 with no wtFGF1 as a control with cell counts of 3T3 fibroblast cells from day 0 to day 3.

FIG. 8B illustrates a cell proliferation assay of the released wtFGF1 from GEL900 and GEL800 with calculated active released wtFGF1 as a function of time.

FIG. 9A illustrates the synthesis of the Poly(acrylic acid)-co-poly(oligoethylene glycolmonoacrylate)-co-poly(N-isopropyl acrylamide) (PAA-co-POEGA-co-PNIPAM). The reaction involves an aqueous free radical polymerization of AA, OEGA (M.W. 480), and NIPAM with the cross-linking agent MBAm initiated by APS.

FIG. 9B shows when after the polymer network is formed, the PAA-co-POEGA-co-PNIPAM polymer will be swollen in buffered saline solution containing 0.1 wt. % Borax (antibacterial agent), forming the injectable hydrogels.

FIG. 10 shows the controlled release of wtFGF-1 from different formulations of injectable anionic PAA-co-POEGA-co-PNIPAM hydrogels with the release was done in physiological conditions at pH 7.4.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure or system. Further, the terms and phrases used herein are not intended to be limiting, but rather to provide an understandable description of the invention.

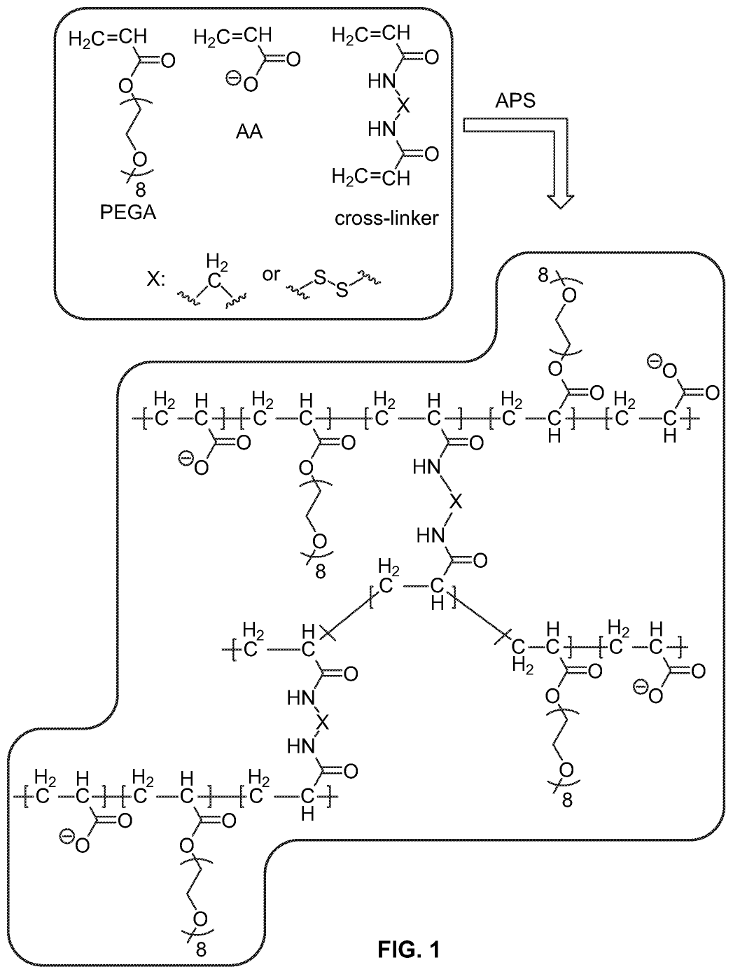

In other embodiments, the present involves the synthesis of the injectable hydrogels that consists of a two-step procedure (FIG. 1). The initial step involves an aqueous free radical polymerization of AA and OEGA (M.W. 480) with a cross-linking agent such as MBAm or BAC. The reaction was initiated by APS to form PAA-co-POEGA polymeric network. The second step is to let the PAA-co-POEGA polymeric network swell in a buffered solution such as a saline solution with 0.1 wt. % Borax (antibacterial agent), forming the injectable hydrogels. Five formulations were synthesized as listed in Table 1. It was determined that the fluidity decreased with increased AA component in the polymer chain. The chain length of the copolymer increased with increased weight percent of AA in the polymerization reaction. The longer the polymer chain, the stronger were the intermolecular interactions between the polymer chains leading to the distinctive difference in physical properties of the resulting hydrogels.

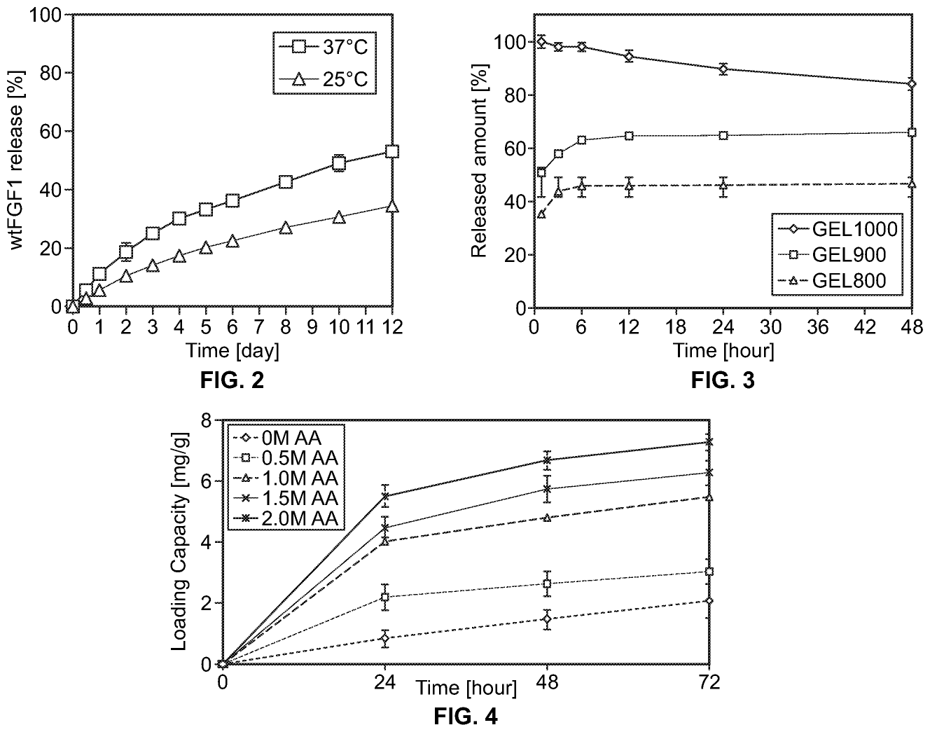

By increasing the concentrations of cross-linker and monomers, the hydrogels gained mechanical strength and became less fluidic. This property of hydrogel helped shaping of the hydrogel as pads or disks as shown in FIG. 2. The pad or disk hydrogel formulation is listed in Table 2.

TABLE 1

| | | POEGA/AA | Mon./MBAm | MBAm | | Reaction | Reaction | Total | Borax |
|---|---|---|---|---|---|---|---|---|---|
| | POEGA/AA | (mol/mol, | (mol/mol, | cont. | APS cont. | Temp | Time | Volume | content |
| Sample | (w/w, mg) | mmol) | mmol) | (mM) | (w/v, ‰) | (° C.) | (min) | (mL) | (w/v, %) |
| GEL1000 | 1000/0 | 2.08/0.00 | 2.08/0.1 | 10 | 1.5 | 70 | 30 | 10 | 0.1 |
| GEL950 | 950/50 | 1.98/0.69 | 2.67/0.1 | 10 | 1.5 | 70 | 15 | 10 | 0.1 |
| GEL900 | 900/100 | 1.88/1.39 | 3.26/0.1 | 10 | 1.5 | 70 | 5 | 10 | 0.1 |
| GEL850 | 850/150 | 1.77/2.08 | 3.85/0.1 | 10 | 1.5 | 70 | 5 | 10 | 0.1 |
| GEL800 | 800/200 | 1.67/2.78 | 4.44/0.1 | 10 | 1.5 | 70 | 5 | 10 | 0.1 |

TABLE 2

| C_{POEGA} (M) | C_{AA} (M) | C_{bis} (mM) | V(mL) | C_{APS} (mM) | T (° C.) |
|---|---|---|---|---|---|
| 0.5 | 0 | 50 | 1 | 50 | 70 |
| 0.5 | 0.5 | 50 | 1 | 50 | 70 |
| 0.5 | 1 | 50 | 1 | 50 | 70 |
| 0.5 | 1.5 | 50 | 1 | 50 | 70 |
| 0.5 | 2 | 50 | 1 | 50 | 70 |

For the injectable hydrogels, a model protein, lysozyme, was mixed with the hydrogels, to demonstrate the withholding capability of the hydrogels. The hydrogel-protein mixtures were placed in PBS and the released amount of lysozyme was monitored by fluorescence spectroscopy up to 48 hours (FIG. 3). Within the first hour, lysozyme was completely released from GEL1000, made from pure POEGA, while only 50% and 35% of lysozyme was released from GEL900 and GEL800, respectively. After 12 hours, the release reached equilibrium wherein 40% and 55% of lysozyme remained in GEL900 and GEL800, respectively suggesting a potential for sustainable release if placed on the wound site. As lysozyme is positively charged at physiological conditions, the increased concentration of AA in the copolymer will enhance the electrostatic interaction(s) between the polymeric network and positively charged proteins and consequently facilitates retention in the hydrogel for prolonged periods of time.

For the disk hydrogel, the loading capacity was assessed using the positively charged lysozyme (FIG. 4). The amount of lysozyme loaded in the hydrogels increased with increased AA concentration in the copolymer is the result of the electrostatic interaction(s) between negatively charged AA and lysozyme.

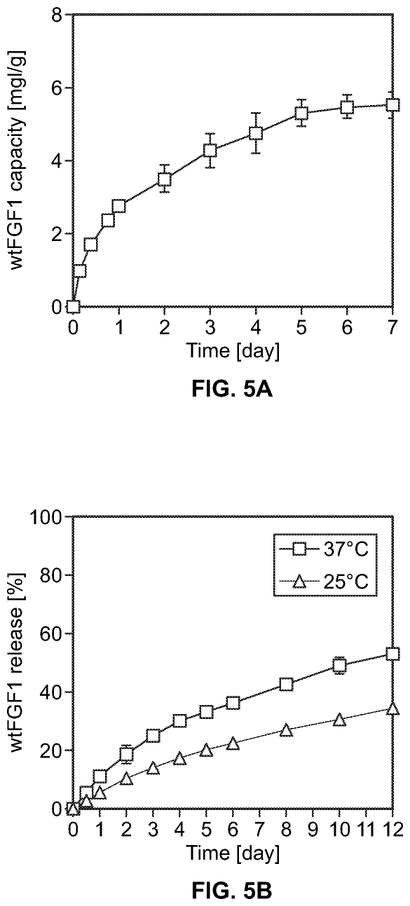

The wtFGF1 was further used to demonstrate the loading and releasing capacity of the disk hydrogel at a 1:1 molar ratio of AA/OEGA (FIG. 5). The loading capacity of disk hydrogels can reach ˜6 mg/g within a week similar to that of lysozyme. The release of wtFGF1 was very slow and temperature-dependent. Over the course of 12 days, ˜50% and ˜30% wtFGF1 was released from the hydrogel at 37° C. and 25° C., respectively.

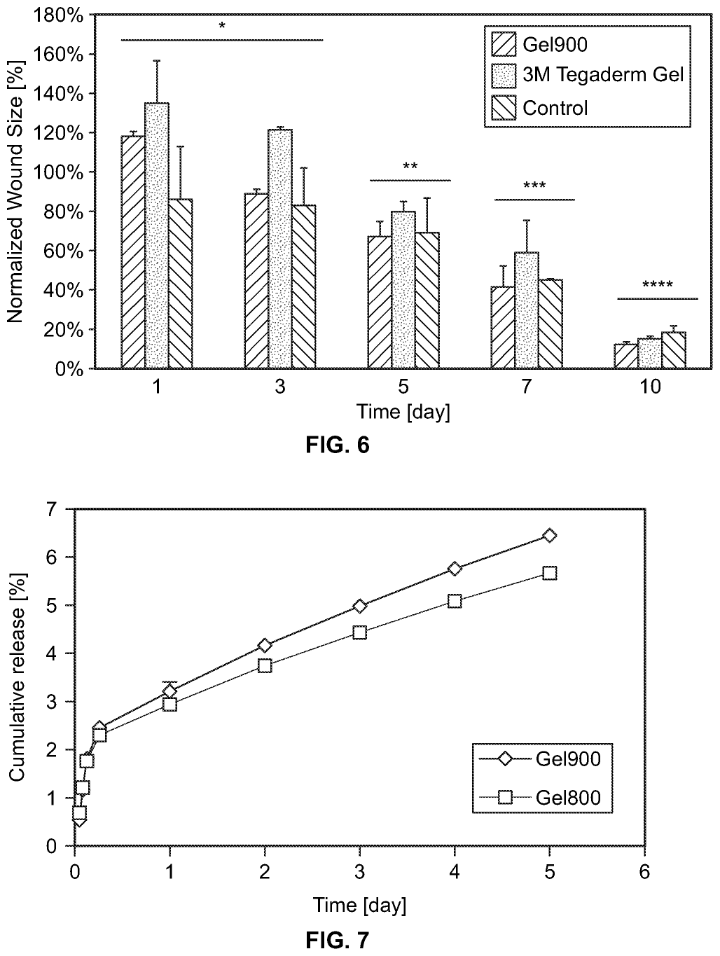

Since the viscosity of GEL900 is comparable to that of the commercially-available 3M Tegaderm injectable hydrogel, it was further considered as a candidate for in vivo studies. The biocompatibility of GEL900 was evaluated using a mouse model of excisional wound healing. FIG. 6 shows the wound size comparison after individual application of GEL900 and the commercially-available 3M Tegaderm Gel. On day 1, wounds treated with the GEL900 and 3M Tegaderm Gel increased in size by 20 to 30%. By day 3, the wounds applied with GEL900 began to decrease in size relative to the 3M Tegaderm Gel. By day 10, wounds treated with GEL900 had nearly closed, indicating biocompatibility. Similar to the control group without gel treatment, significant differences were observed between the day 1 or day 3 and the later days (i.e., day 5, 7, or 10) for both groups treated with gels. GEL900 appeared to be comparable to the 3M Tegaderm Gel during healing and neither of the gels was observed to delay the normal healing process.

In other embodiments, the present invention provides anionic hydrogels based on poly(acrylic acid)-co-poly(oligoethylene glycol monoacrylate) (PAA-co-POEGA) for protein delivery. The hydrogel comprises an injectable hydrogel prepared in two steps, one-pot method which involves an aqueous free radical polymerization, followed by sequential steps to allow the swelling of copolymer into the hydrogels. The hydrogel may then be retained in a number of different predetermined shapes such as a disk, cylinder, sphere and other shapes known to those of skill in the art.

A disk shape, redox-induced degradable hydrogel may also be prepared from the same monomer but crosslinked by redox-responsive cross-linking reagent. In a preferred embodiment, the injectable hydrogel comprises a copolymer comprising poly(oligoethylene glycol monoacrylate), acrylic acid, N,N′-methylenebis(acrylamide), sodium tetraborate and phosphate-buffered saline wherein a mass percentage of the components comprises: poly(oligoethylene glycol monoacrylate) ranging from 8%-10%; acrylic acid ranging from 2%-0%; N,N′-methylenebis(acrylamide) was fixed to 0.154%; sodium tetraborate was fixed to 0.1%; and phosphate-buffered saline up to 100%. The injectable hydrogel may also have a molecular weight of poly(oligoethylene glycol monoacrylate) of 480 g/mol.

In other aspects, the present invention provides a method of producing an injectable hydrogel comprising the steps of providing a) Poly(oligoethylene glycol monoacrylate), acrylic acid and N,N′-methylenebis(acrylamide) and water in a Schleck tube or other suitable container which may be bubbled with N₂ gas for 30 min before sealing the container which is followed by one or more and preferably three evacuate-refill cycles with Ar to remove the dissolved oxygen. The reaction solution may then be incubated at 70° C. for 30 min. After incubation, an initiator may be added under Ar flow, followed by another three evacuate-refill cycles with Ar. The reaction was allowed to proceed at 70° C. for 5-30 min under magnetic stirring at a speed of 350 rpm. Immediately after the reaction, the polymerization was terminated by cooling the container. The reaction solution was then dialyzed against water using dialysis membrane. The resulted gel solution was lyophilized into a form of a dry gel. The lyophilized copolymer was swollen PBS containing borax for 24 h to form the injectable anionic hydrogels.

In other steps, the polymerization may be initiated by ammonium persulphate and the molecular weight cut off of the dialysis membrane is 2 kDa. The viscosity of the injectable hydrogel may be controlled by the molar ratio between the total monomer and crosslink agent. The thermal stability of the injectable hydrogel may be controlled by the molar ratio between the total monomer and crosslink agent. In addition, one or more positively charged proteins may also be bonded to the injectable hydrogel by electrostatic interaction and the protein's capacity controlled by the molar concentration of acrylic acid. The positively charged protein release kinetic of the injectable hydrogel may be controlled by the molar concentration of acrylic acid.

In other aspects, the present invention provides a disk shape hydrogel comprising a copolymer comprising poly(oligoethylene glycol monoacrylate), acrylic acid, N,N′-methylenebis(acrylamide) and phosphate-buffered saline; wherein a mass percentage of the components may be as follows: poly(oligoethylene glycol monoacrylate) fixed to 24%; acrylic acid ranging from 0%-14.4%; N,N′-methylenebis(acrylamide) fixed to 0.77%; and phosphate-buffered saline up to 100%. The molecular weight of poly(oligoethylene glycol monoacrylate) may be 480 g/mol.

In other aspects, the present invention provides a method of producing cylindrical shaped hydrogel comprises of the following steps: providing poly(oligoethylene glycol monoacrylate), acrylic acid and N,N′-methylenebis(acrylamide) and water in a container which is bubbled with N₂ gas for 30 min before sealing the container and then adding an imitator. The reaction may be started by heating up to 70° C. for 4 h. The resulting hydrogel is washed preferably with water and allowed to completely swell in PBS for example. When the hydrogel is cylindrical in shape, the swelling ratio may be controlled by the molar ratio between the total monomer and crosslink agent. The polymerization may be initiated by ammonium persulphate.

A positively charged protein may be bonded to the shaped hydrogel by electrostatic interaction. The protein capacity may be controlled by the molar concentration of acrylic acid and the positively charged protein release kinetic of the disk shape hydrogel may be controlled by a competition between total protein capacity of hydrogel and molar concentration of acrylic acid.

In other aspects, the present invention provides a biodegradable disk shape hydrogel comprising a copolymer comprising poly(oligoethylene glycol monoacrylate), acrylic acid, N,N′-bis(acryloyl)cystamine and phosphate-buffered saline. The mass percentage of the components is as follows: poly(oligoethylene glycol monoacrylate) fixed to 24%; acrylic acid ranging from 3.6%-14.4%; N,N′-Methylenebis(acrylamide) fixed to 1.3%; and phosphate-buffered saline up to 100%. The molecular weight of poly(oligoethylene glycol monoacrylate) may be 480 g/mol.

In other aspects, the present invention provides a method of producing a disk shape hydrogel comprising the steps of: poly(oligoethylene glycol monoacrylate), acrylic acid, N,N′-bis(acryloyl)cystamine and water/EtOH mixture in a container was bubbled with N₂ gas for 30 min before sealing the container and adding an imitator with the reaction being started by heating up to 70° C. for 4 h. Next the resulting hydrogels are washed and allowed to swell to completion. In other embodiments, the polymerization may be initiated by ammonium persulphate and the volume ratio of water and EtOH is 3/1. The swelling ratio of the biodegradable disk shape hydrogel can be controlled by the molar ratio between the total monomer and crosslink agent and the positively charged protein may be bonded to the biodegradable disk shape hydrogel.

Other aspects of controlling aspects of the hydrogel include the following: the protein capacity may be controlled by the molar concentration of acrylic acid; the positively charged protein release kinetic of the disk shape hydrogel may be controlled by a competition between total protein capacity of hydrogel and the molar concentration of acrylic acid; and the biodegrade speed of the hydrogel may be controlled by the free thiol concentration.

Controlled Release of wtFGF1 from Injectable Anionic Hydrogels.

In another embodiment, 200 mg of injectable gel was mixed with 100 μL of 3.0 mg/mL wtFGF1 and stored at 4° C. overnight. The mixture was loaded into a transwell membrane plates insert (3 μm pore size) with a polyester membrane and polystyrene plates. The insert was then immersed in 1.5 mL of 1×PBS releasing media for controlled release at room temperature. At desired time points, 1 mL of the released medium was sampled and replaced by an equal volume of fresh medium to maintain a constant volume. The medium was then analyzed using fluorescence spectrophotometer. Then cumulative of released wtFGF1 was calculated from wtFGF1 calibration curve at 309 nm emission wavelength. The process was repeated three times and the results were reported as average values with standard deviations. The concentration of the FGF in each transwell is determined according to the measured fluorescence intensity using a standard curve. Unlike the lysozyme, the results in FIG. 7 indicate that there is little release of the wtFGF1 from the gels. This is possibly due to the strong electrostatic interaction of the wtFGF1 with the PAA component in the gel.

Cell Proliferation Assay for Bioactivity of Released wtFGF1.

Bioactivity of FGF was evaluated after release activity from PAA-co-POEGA hydrogels to ensure sustained activity. 3T3 fibroblast cells were grown to 80-90% confluency. Cell proliferation activity of released wtFGF1 was performed by incubating 10,000 cell/well with 50 ng/mL of released wtFGF1 in serum-supplemented medium. Cell proliferation assay was determined by CellTilter-Glo luminescent cell viability assay.

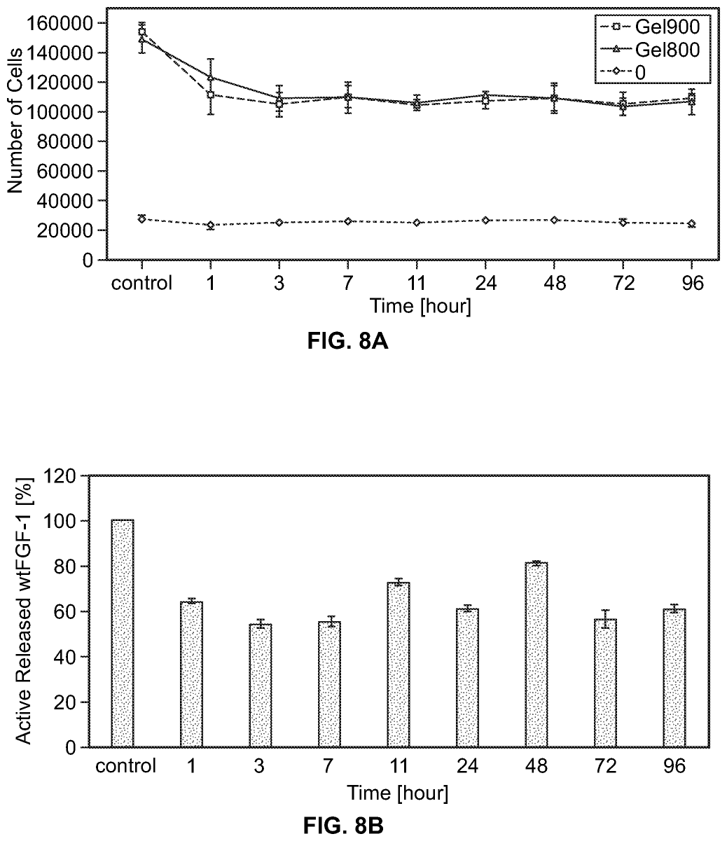

FIGS. 8A and 8B show the cell proliferation assay of the released wt.FGF1 from GEL900 and GEL800, respectively. Compared to the same amount of freshly-prepared wtFGF1, the data shows about 60% of released wtFGF1 was active.

Poly(acrylic acid)-co-poly(oligoethylene glycolmonoacrylate)-co-poly(N-isopropyl acrylamide) Injectable Anionic Hydrogels for Controlled Release of wtFGF1 release.

Synthesis of PAA-co-POEGA-co-PNIPAM injectable hydrogels.

Each monomer was purified prior to the polymerization using the following methods. Oligoethylene glycol monoacrylate (Mₙ=480, OEGA) was purified through a basic Al₂O₃ column. Acrylic acid (AA) was purified through a neutral Al₂O₃ column. N-isopropyl acrylamide (NIPAM) was recrystallized from hexane. The polymer was synthesized by copolymerization of the three monomers, AA, EGA, and NIPAM in an aqueous solution initiated by ammonium persulfate (APS). During the free radical polymerization, the monomers were cross-linked by N, N′-methylenebis (acrylamide) (MBAm). Typically, 10 mL aqueous solution containing a total amount of 1000 mg of OEGA, PAA and PNIPAM, and 15.4 mg MBAm (10 mM) was added to a 50-mL Schleck tube flask. The weight ratios of the three monomers in different formulation are listed in Table 3. The flask was bubbled with N₂ gas for 30 min before it was sealed. The flask was then subjected to three evacuate-refill cycles with Argon (Ar) to eliminate the dissolved oxygen. The reaction was incubated at 70° C. for 30 min. After incubation, 200 μL aqueous solutions of APS (15 mg) were added to the tube under Ar flow, followed by another three evacuate-refill cycles with Ar. The reaction could proceed at 70° C. for 5 min under magnetic stirring at 350 rpm. Immediately after the reaction, the polymerization was terminated by cooling the tube in an ice-water bath.

TABLE 3
Different formulations of PAA-co-POEGA-
co-PNIPAM injectable hydrogels.

| | Total | | | | |
|---|---|---|---|---|---|
| | Volume | APS | MBAM | OEGA/AA/NIPAM | Temperature |
| Sample ID | (mL) | (mg) | (mM) | w/w/w, mg | (° C.) |
| GEL 1 | 10 | 15 | 10 | 700/200/100 | 70 |
| GEL 2 | 10 | 15 | 10 | 900/50/50 | 70 |
| GEL 3 | 10 | 15 | 10 | 900/75/25 | 70 |
| GEL 4 | 10 | 15 | 10 | 800/150/50 | 70 |
| GEL 5 | 10 | 15 | 10 | 800/100/100 | 70 |
| GEL 6 | 10 | 15 | 10 | 800/50/150 | 70 |

PAA-co-POEGA-co-PNIPAM Gel.

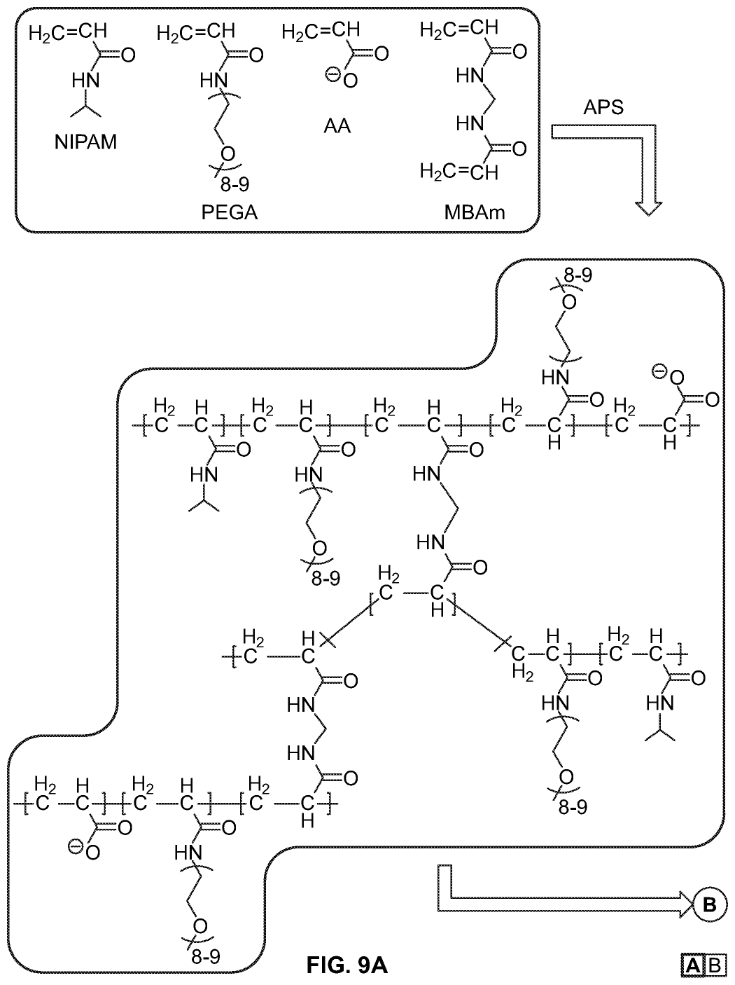

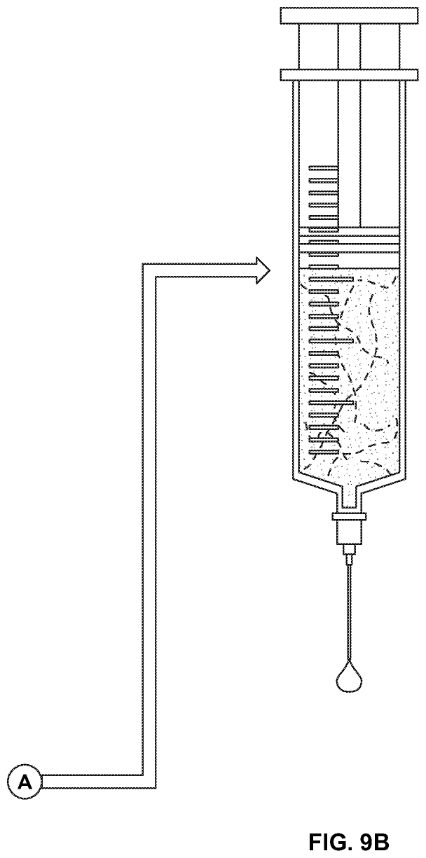

The newly-developed anionic injectable hydrogels were prepared from the copolymer PAA-co-POEGA-co-PNIPAM, as shown in FIG. 9. The incorporation of the NIPAM in the polymer network may space out the negative charges in the two-component PAA-co-POEGA polymer. This may facilitate the release kinetics of wtFGF1 while maintaining the viscosity of the injectable gels for wound healing applications. In addition, the PNIPAM can response to heat changes that may be used for other applications that would involve with the use of heat as a tuning knob.

wtFGF1 Control Release Study.

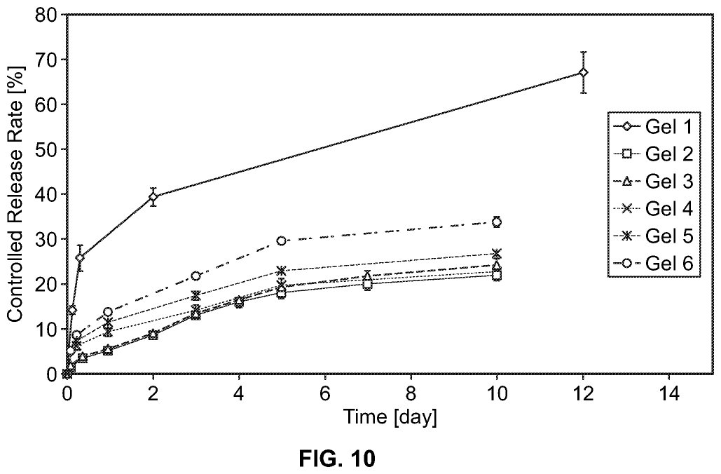

The release of wtFGF1 was significantly increased using PAA-co-POEGA-co-PNIPAM gels compared to PAA-co-POEGA, as shown in FIG. 10. The decrease of OEGA percentage in the formulation significantly increase the release kinetics of wtFGF1 from the gel. The release of wt-FGF1 could reach nearly 100% at 25 days (data not shown here). In addition, the decrease of the ratio AA/PNIPAM will increase the release kinetics of wtFGF1 from the gel providing a fine modulation of the wtFGF1 release.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above described embodiments, methods, and examples, but by all embodiments and methods within the scope and spirit of the disclosure.

What is claimed is:

1. Anionic hydrogels based on poly(acrylic acid)-co-poly(oligoethylene glycol monoacrylate) (PAA-co-POEGA) for protein delivery, the hydrogel types comprises: a hydrogel prepared in two steps, one-pot method which involves an aqueous free radical polymerization, followed by sequential steps to allow the swelling of copolymer into the hydrogels.

2. The hydrogel of claim 1 wherein the hydrogel is an injectable hydrogel.

3. The hydrogel of claim 1 wherein the hydrogel is a disk shape hydrogel.

4. A method of producing an hydrogel comprising the steps of: providing a) poly(oligoethylene glycol monoacrylate), acrylic acid and N,N′-methylenebis(acrylamide) and water in a container bubbled with a gas before sealing said container; b) incubating; c) adding an initiator; d) performing one or more evacuate-refill cycles; e) terminating the polymerization by cooling; f) dialyzing against water using dialysis membrane; g) lyophilizing into a form of dry gel; and h) swelling said gel to form and injectable anionic hydrogel.

5. The method of claim 4 wherein the hydrogel is an injectable hydrogel.

6. The method of claim 4 wherein the hydrogel is a disk shape hydrogel.

7. The method of claim 4, wherein the polymerization is initiated by ammonium persulphate.

8. The method of claim 4, wherein the molecular weight cut off of the dialysis membrane is 2 kDa.

9. The method of claim 5 further including the step of controlling the viscosity of the injectable hydrogel by controlling the molar ratio between the total monomer and a crosslink agent.

10. The method of claim 4 further including the step of controlling the disk shape hydrogel by controlling the molar ratio between the total monomer and a crosslink agent.

11. The method of claim 5 further including the step of bonding one or more positively charged proteins to the injectable hydrogel by electrostatic interaction.

12. The method of claim 11, wherein the protein capacity is controlled by the molar concentration of acrylic acid.

13. The method of claim 11, wherein the positively charged protein release kinetic of the injectable hydrogel according is controlled by the molar concentration of acrylic acid.

14. The disk shape hydrogel according to claim 3 comprising a copolymer comprising poly(oligoethylene glycol monoacrylate), acrylic acid, N,N′-methylenebis(acrylamide) and phosphate-buffered saline; wherein a mass percentage of the components comprises of: poly(oligoethylene glycol monoacrylate) fixed to 24%; acrylic acid ranging from 0%-14.4%; N,N′-methylenebis(acrylamide) fixed to 0.77%; and phosphate-buffered saline up to 100%.

15. The method of claim 6 wherein the disk shape hydrogel comprising a copolymer comprising poly(oligoethylene glycol monoacrylate), acrylic acid, N,N′-methylenebis(acrylamide) and phosphate-buffered saline; wherein a mass percentage of the components comprises of: poly(oligoethylene glycol monoacrylate) fixed to 24%; acrylic acid ranging from 0%-14.4%; N,N′-methylenebis(acrylamide) fixed to 0.77%; and phosphate-buffered saline up to 100%.

16. The disk shape hydrogel according to claim 14, wherein the molecular weight of poly(oligoethylene glycol monoacrylate) is 480 g/mol.

17. The hydrogel of claim 1 wherein the hydrogel includes MBAm.

18. The hydrogel of claim 1 wherein the hydrogel includes POEGA.

19. The hydrogel of claim 1 wherein the hydrogel includes PAA.

20. The hydrogel of claim 1 wherein the hydrogel includes PNIPAM.
